(19) United States
(12) Patent Application Publication
(10) Pub. No.: US 2007/0027175 A1
(43) Pub. Date: Feb. 1, 2007

(54) Antineoplastic activities of ellipticine and its derivatives

(76) Inventors: John Shaughnessy JR., Roland, AR (US); Erming Tian, Little Rock, AR (US)

Correspondence Address: Benjamin Aaron Adler;ADLER & ASSOCIATES, 8011 Candle Lane, Houston, TX, 77071, US

(21) Appl. No.: 11/494,335

(22) Filed: Jul. 27, 2006

Related U.S. Application Data
(60) Provisional application No. 60/702,944, filed on Jul. 27, 2005.

(51) Int. Cl. A61K 31/4745

(52) U.S. Cl. 514/283

(57) ABSTRACT

The present invention describes selective cell growth inhibition of myeloma cells by ellipticine derivatives, 9-methoxy ellipticine and 9-dimethyl amino-ethoxy ellipticine. The cell growth inhibition efficacy was highest for 9-dimethyl amino-ethoxy ellipticine among the ellipcitine derivatives tested. The cell toxicity of 9-dimethyl amino-ethoxy ellipticine was selective for myeloma cells and did not kill normal cells in the effective antineoplastic dose range. 9-dimethyl amino-ethoxy ellipticine was superior to existing antimyeloma drugs, Adriamycin® and Etoposide in eliciting early and better cell growth inhibition response.

### CROSS REFERENCE TO RELATED APPLICATION

This non-provisional application claims benefit of provisional application U.S. Ser. No. 60/702,944 filed on Jul. 27, 2005, now abandoned.

### FEDERAL FUNDING LEGEND

This invention was produced using funds obtained through grant CA55819-10 from the National Institutes of Health. Consequently, the federal government has certain rights in this invention.

### BACKGROUND OF THE INVENTION

1. Field of the Invention

The present invention relates generally to the field of cancer therapeutics. More specifically, the present invention relates to the derivatives of the plant alkaloid, ellipticine, as new therapeutic agents for treating cancer.

2. Description of the Related Art

Ellipticine (5,11-Dimethyl-6H-Pyridol[4,3]carbazole, MW+246.3), an alkaloid isolated from Apocyanaceae plants, is a topoisomerase II inhibitor that induces topo II dependent DNA cleavage. Ellipticine has been shown to exhibit-significant anti-tumor and anti-HIV activity (Stiborova et al., 2001). The antineoplastic mechanism of ellipticine comprised formation of covalent DNA adducts, which was mediated by human cytochrome P450 (CYP). Additionally, the same study also elucidated the metabolites responsible for DNA binding (Stiborova et al., 2004).

Furthermore, derivatives of ellipticine, NSC 69187 (ellipticine-9-methoxy-, MW=276.0) and NSC 338258 (ellipticine, 9-dimethyl amino-ethoxy-, MW=406.0) have been identified as new therapeutic agents for cancer and AIDS by the Developmental Therapeutics Program (DTP) and the National Cancer Institute (NCI). The NCI in vivo screening data indicates that the dosages for animals surviving the toxicity of the compounds were 37.5 mg/kg body weight and 25.0 mg/kg body weight for NSC 69178 and NSC 338258 respectively. In a range of 9.37 mg/kg to 75.0 mg/kg in vivo antitumor activity of NSC 69178, more than 90% of animals (BDF1 mice) survived the tumor model and dose toxicity at the endpoint of 60 days.

With regard to treatment of cancer, there are some cancers that respond initially to most chemotherapeutics but may develop multi-drug resistance and prove fatal. For instance, multiple myeloma remains a uniformly fatal malignancy due to development of multi-drug resistance despite initial response to most chemotherapeutics (Barlogie and Shaughnessy, 2004). Multiple myeloma evolves in the hematopoietic system, which selectively encodes this malignancy to retain its inherent ability to deactivate the patient's immune surveillance and suppression. During myeloma oncogenesis, genetic aberrations to myeloma cells also bolster future acquired resistance to therapy. In multiple myeloma, a collection of genetic mutations correlate with poor prognosis (Shaugnessy et al., 200; Desikan et al., 2004; Shaughnessy et al., 2001, Sawyer et al., 2005). These mutations are necessary for adapting the bone marrow environment not only to support myeloma growth and proliferation, but also to allow myeloma cells to evade apoptotic processes induced by therapeutic agents. Anti-myeloma therapeutic agents eventually fail for a range of reasons, including their lack of specificity, rapid metabolism, and molecular modifications in cancer cells. Thus, there is a tremendous need to identify novel anti-myeloma agents that efficiently overcome these confounding factors.

Recent gene expression-profiling study of 351 specimens from patients newly diagnosed with myeloma demonstrated that, in myeloma patients, poor prognosis was associated with a frequently amplified and translocated locus at chromosome arm 1q21 (Sawyer et al., 2005). Expression of CKS1B, which lies within this locus and controls several aspects of cell cycle progression, was significantly related to the prognoses of myeloma in these patients (Shaugnessy, 2005). Despite this, there is lack of therapeutics that target cells with high levels of CKS1B expression.

Thus, prior art is deficient in drugs target cells that express high levels of CKS1B. Specifically, the prior art is deficient in effective treatment of for myeloma. The present invention fulfills this long-standing need and desire in the art.

### SUMMARY OF THE INVENTION

The present invention is directed to a method of treating myeloma in an individual. Such a method comprises administering a pharmcologically effective dose of ellipticine, a derivative of ellipticine or a combination thereof.

Alternatively, the present invention is also directed to a method of treating myeloma in an individual. Such a method comprises administering a pharmacologically effective dose of a topoisomerase II inhbitior, where the inhibitor induces cell cyle arrest of myeloma cells, induces apoptosis of myeloma cell, overcomes acquired drug resistance or a combination thereof without affecting the viability of normal cell. This results in treatment of myeloma in the individual.

The present invention is further directed to a method of inhibiting growth of a myeloma cell. Such a method comprises contacting the myeloma cell with ellipticine, a derivative thereof or a combination thereof.

The present invention is still further directed to a method of inducing apoptosis of a myeloma cell. Such a method comprises contacting the myeloma cell with ellipticine, a derivative thereof or a combination thereof such that said contact activates caspase 9, thereby inducing apoptosis of the myeloma cell.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 shows the anti-myeloma activity of the four ellipticine derivatives, 19942 J/4 (▴), 130789 U/1 (X), 316458 K/4 (*), 338258 G/1 (▪) and 630740 U/1 (I) for the myeloma cell line Ark. The dosage of each derivative was set at 0.5 mg/ml. A cell culture medium control (●) and 0.5% DMSO control (▮) was maintained in the experiment. The cell viability was tested each day over a period of five days using the CellTiter-Glo luminescent cell viability assay kit from Promega Co.

FIG. 2 shows the anti-myeloma activity of the four ellipticine derivatives, 19942 J/4 (▴), 130789 U/1 (X), 316458 K/4 (*), 338258 G/1 (●) and 630740 U/1 (I) for the myeloma cell line kms 11. The dosage of each derivative was set at 0.5 mg/ml. A cell culture medium control (●) and 0.5% DMSO control (▮) was maintained in the experiment. The cell viability was tested each day over a period of five days using the CellTiter-Glo luminescent cell viability assay kit from Promega Co.

FIG. 3 shows the anti-myeloma activity of the four ellipticine derivatives, 19942 J/4 (▴), 130789 U/1 (X), 316458 K/4 (*), 338258 G/1 (▪) and 630740 U/1 (I) for the myeloma cell line RPMI8226. The dosage of each derivative was set at 0.5 mg/ml. A cell culture medium control (●) and 0.5% DMSO control (▮) was maintained in the experiment. The cell viability was tested each day over a period of five days using the CellTiter-Glo luminescent cell viability assay kit from Promega Co.

FIG. 4 shows the anti-myeloma activity of the four ellipticine derivatives, 19942 J/4 (▴), 130789 U/1 (X), 316458 K/4 (*), 338258 G/1 (▪) and 630740 U/1 (I) for the myeloma cell line U266. The dosage of each derivative was set at 0.5 mg/ml. A cell culture medium control (●) and 0.5% DMSO control (▮) was maintained in the experiment. The cell viability was tested each day over a period of five days using the CellTiter-Glo luminescent cell viability assay kit from Promega Co.

FIG. 5 shows the invitro comparison of ellipticine (EPE), NSC 69187 (EPE-Der6) and NSC 338258 (EPE-Der3) with respect to their anti-myeloma activity for the four myeloma cell lines, ARK, CAG, RPM18226 and U266. The dosage of each derivative was set at 2, 0.2 and 0.02 mM. A cell culture medium control and 0.5% DMSO control was maintained in each experiment. The cell viability was tested each day over a period of five days using the CellTiter-Glo luminescent cell viability assay kit from Promega Co.

FIG. 6 shows the comparison of ellipticine analog NSC 338258 (▴) with existing anti-myeloma drugs, Adriamycin® ( [symbol] ) and Etoposide (♦). A cell culture medium control and 0.5% DMSO control was maintained in each experiment. The results obtained from these controls are pooled together in the figure (●). The dose of each compound was set at 0.2 mM. The cell viability was tested each day over a period of five days using the CellTiter-Glo luminescent cell viability assay kit from Promega Co.

FIG. 7 shows the viability of normal fetal bone mesenchymal cells (FB MSC) when contacted with NSC 338258 (EPE-Der3) in the concentration range of 0.01-1.0 mM. The cell viability was tested each day over a period of five days using the CellTiter-Glo luminescent cell viability assay kit from Promega Co.

FIG. 8A shows the overall cell growth inhibition by NSC 388258. Five non myeloma cancer cell lines, HEL (▮), HL-60 (▴), k562 (X), MEG01 ( [symbol] ) and THP1 (▪) were exposed to NSC 388258 over a period of 5 days. The dose was set at 0.2 mM. A cell culture medium control and 0.5% DMSO control was maintained in each experiment. The results obtained from these controls are pooled together in the figure (●). The cell viability was tested each day over a period of five days using the CellTiter-Glo luminescent cell viability assay kit from Promega Co.

FIG. 8B shows the overall cell growth inhibition by NSC 388258. Seven non myeloma cancer cell lines, Hela (▮), G401 (▴), Du145 (X), sw480, (*) sw620 (▪), SaoS2 (I) and MG63 (−) were exposed to NSC 388258 over a period of 5 days. The dose was set at 0.2 mM. A cell culture medium control and 0.5% DMSO control was maintained in each experiment. The results obtained from these controls are pooled together in the figure (●). The cell viability was tested each day over a period of five days using the CellTiter-Glo luminescent cell viability assay kit from Promega Co.

FIG. 9 shows the chemical structures of ellipticine and EPED3. NSC 338258 (EPED3, right) is a water-soluble derivative of ellipticine with the modification at position 9.

FIGS. 10A-10B show that EPED3 has high anti-myeloma efficacy. U266 cell line was one of 12 myeloma cell lines used in screening of anti-myeloma agents. In FIG. 10A, cells were treated with 14 ellipticine derivatives (0.5 mM) obtained from the DTP; control cells were untreated. Ellipticine derivatives are coded with NSC identifiers. For 5 days, cell viability was measured every 24 hours using CellTiter-Glo Luminescent Assay. EPED3 exhibits drastic cytotoxic activity on U266 cells. In FIG. 10B, U266 cells, along with six other myeloma cell lines, were co-cultured in direct contact with human fetal bone mesenchymal cells and treated with EPED3 (0.5 mM and 2 mM) or Velcade (0.5 mM); control cells were untreated. Cell proliferation was assessed by MTT assay every 24 hours for 5 days. At 2 mM, EPED3 exhibited similar reductions in cell proliferation efficacy as 0.5 mM Velcade. Error bars indicate standard deviation.

FIGS. 11A-11F compare the morphological features of EPED3 and Velcade induced apoptosis in myeloma cells and normal mesenchymal cells. JJN3 myeloma cell line and human fetal bone mesenchymal cells were co-cultured without direct contact between cell types. EPED3 (2 mM) and Velcade (0.5 mM) were added to the cultures; control cells were untreated. Live cell cultures were photographed 3 days after initiating treatment (400× magnification under Olympus invert-microscope with RT-Color SPOT digital camera and the software, Diagnostic Instrument Inc. Sterling Heights, Mich.). In comparison to cell morphology of non-treated JJN3 cells, myeloma cells were destroyed under both EPED3 (FIG. 11E) and Velcade (FIG. 11F) treatments as the appearance of cell shrinkage, and the formation of phagozytosed apoptotic bodies (arrows); in comparison to no treatment, mesenchymal cells were also destroyed by Velcade treatment as the appearance of cell shrinkage, and the formation of phagozytosed apoptotic bodies (arrows) (FIG. 1C) but not by EPED3 treatment (FIG. 11B).

FIGS. 12A-12B show dose response of myeloma cells to common anti-myeloma agents used in clinics. U266 was one of 4 myeloma cell lines exposed to a panel of anti-myeloma agents in 2-fold titration (6.4-0.1 mM). MTT assays were performed to assess cell proliferation inhibition for 24 hours (FIG. 12A) and 48 hours (FIG. 12B). Without co-culture condition, Velcade exhibits a great efficiency on killing U266 cells (less than 0.1 μM). EPED3 stopped cell proliferation at 0.2 μM, and exhibited better efficacy on reduction of cell metabolic activity at 0.4 μM or greater. Error bars indicate standard deviation.

FIGS. 13A-13B show dose response of RPMI 8226 and 8226/Dox1V myeloma cells to EPED3 (FIG. 13A) or Dox (FIG. 13B). Cells were incubated for 96 hours with drug concentrations as indicated and analyzed by MTT assays; linear regression analysis was used to determine the IC₅₀ for each drug (EPED3, FIG. 13A; Dox, FIG. 13B) and each cell line (RPMI8226, squares; 8226/Dox1V, circles). Data are presented as the mean of four independent experiments. While RPMI8226/Dox1V showed no significant resistance to EPED3, mean IC₅₀ of RPM18226 is 150.3 nM and of 8226/Dox1V is 131.3 nM to EPED3 (p=0.7). In response to Dox, the mean IC₅₀ of RPMI 8226 is 40 nM, and mean IC₅₀ of 8226/Dox1V is 293.5 nM (7.3-fold higher resistance than the parental line, p<0.0001).

FIGS. 14A-14L show flowcytometry analyses of cell viability and cell cycle arrest under EPED3 and Velcade treatments. U266 cells were co-cultured without contact with human fetal bone mesenchymal cells. Cells were untreated (Control; FIGS. 14A, 14D, 14G, 14J) or exposed to 2 mM EPED3 (FIGS. 14B, 14E, 14H, 14K) or 0.5 mM Velcade (FIGS. 14C, 14F, 14I, 14L) for 12 hours and then harvested. Pan-caspase inhibitor Z-VAD-FMK (50 mM) was also added to co-cultures (FIGS. 14D-14F and FIGS. 14J-14L) to block caspase-dependent apoptosis and then analyzed for cell cycling arrest. Percent cells in G₀+G₁ and G₂+M phases were measured by the peaks of DNA helix and are gated with each graph (FIGS. 14A-14F). Percent cells with fragmented DNA are indicated as c % in each graph. Cells were also analyzed for apoptotic status (FIGS. 14G-14L) using Coulter Annexin-V FITC/7-AAD kit. Untreated cells (Control; FIGS. 14G and 14J) were maintained at high viability (90%). Early apoptotic cells were gated at G4, and necrotic cells were gated at G2; percent cells in each population are indicated with each graph. The arrows indicate percent viable cells ingested EPED3, whose fluorescent property was captured at channel FL4 (FIGS. 14H, 14K).

FIGS. 15A-15C show fluorescent images of EPED3 endoplasmic distribution and induction of apoptosis, which was also detected by Western Blot. U266 cells (10⁶ cells/ml) were co-cultured without contact with human fetal bone mesenchymal cells and treated with EPED3 (2 mM) or Velcade (0.5 mM). For analysis by fluorescence microscopy, nuclei were stained with DAPI (blue), mitochondria with MitoTracker Red CMXRos (red), and cytochrome c with FITC-conjugated secondary antibodies (green); EPED3 is excited by ultraviolet light (wavelength<500 nm) and was visualized as gold.

FIG. 15A shows cells photographed under fluorescence microscopy at the time of treatment and 30 minutes, 1.5 hours, and 6 hours thereafter. The appearance of large cytoplasmic vacuoles (arrows) and diminished mitochondria in cells treated with EPED3 indicate ongoing apoptosis. FIG. 15B shows cells treated with EPED3 and Velcade were examined after 6 hours for lyses of mitochondria and release of cytochrome c (green). Cytochrome c disassociation with mitochondria also indicates initiation of the intrinsic apoptotic pathway. FIG. 15C compares the expression of caspases in myeloma cell lines (JJN3, L363, OPM2, and U266) were co-cultured without contact with human fetal bone mesenchymal cells and treated with EPED3 (2 mM) or Velcade (0.5 mM) for 6 hours. Protein extracts (100 mg) were analyzed by Western blotting, using goat anti-human Caspase-3, -8, and -9 polyclonal antibodies. Inactivation of Caspase-8 indicates that the extrinsic apoptotic pathway was not induced by EPED3 or Velcade treatments. In contrast, both EPED3 and Velcade treatments initiated the intrinsic pathway, which releases cytochrome c to induce rapid cleavage of Caspase-9 into its activated form. The consequent Caspase-3 activation indicates activity of the intracellular proteolytic cascade. Abbreviations: C=Medium control, E=EPED3, V=Velcade.

### DETAILED DESCRIPTION OF THE INVENTION

The present invention is directed to the use of ellipticine and its derivatives in the treatment of cancer cells such as myeloma cells. It has been shown herein that the ellipticine derivatives, for instance NSC 338258 (EPED3) and NSC 69178 were cytotoxic to myeloma cell lines. These ellipticine derivatives were identified as strong inhibitors of cell growth with the effective invitro dosage of 0.2 μM. Additionally, further comparison of the cell growth inhibitory effects showed NSC 33828 to be more toxic to all cell lines among all other compounds that were tested. Results summarized in Table 1 show that both the ellipticine derivatives have better anti-myeloma activity compared to ellipticine. It shows that of the two derivatives tested herein, NSC 338258 was better than NSC 69187 with respect to anti-myeloma activity. These results indicate that ellipticine, NSC 69187 and NSC 338258 can be used in the treatment of myeloma.

Table 1 shows the cell viability over a period of five days as determined by the CellTiter- Glo luminescent assay kit fro Promega Co.

| | Ark | CAG | RPMI8226 | U266 |
|---|---|---|---|---|
| Day 1 | | | | |
| Control (Medium) | 116,033 | 199,264 | 104,089 | 32,473 |
| Ellipticine 2 mM | 86,010 | 136,450 | 87,440 | 23,680 |
| Ellipticine 0.2 mM | 125,251 | 196,263 | 107,014 | 32,490 |
| Ellipticine 0.02 mM | 120,709 | 200,429 | 110,290 | 31,950 |
| 69187 2 mM | 14,932 | 88,285 | 21,912 | 7,818 |
| 69187 0.2 mM | 92,678 | 164,436 | 82,834 | 24,729 |
| 69187 0.02 mM | 119,617 | 188,754 | 103,366 | 30,188 |
| 338258 2 mM | 523 | 76,441 | 4,685 | 2,559 |
| 338258 0.2 mM | 1,085 | 103,658 | 3,790 | 5,906 |
| 338258 0.02 mM | 99,050 | 173,140 | 94,826 | 26,346 |
| Day 2 | | | | |
| Control (Medium) | 210,952 | 572,467 | 242,963 | 53,458 |
| Ellipticine 2 mM | 98,439 | 200,722 | 115,263 | 31,682 |
| Ellipticine 0.2 mM | 230,950 | 541,300 | 235,145 | 47,622 |
| Ellipticine 0.02 mM | 208,608 | 557,030 | 244,140 | 47,812 |
| 69187 2 mM | 815 | 58,508 | 2,688 | 3,633 |
| 69187 0.2 mM | 208,050 | 358,735 | 148,198 | 38,799 |
| 69187 0.02 mM | 244,779 | 552,402 | 238,331 | 46,889 |
| 338258 2 mM | 342 | 39,273 | 923 | 691 |
| 338258 0.2 mM | 489 | 105,998 | 2,320 | 1,414 |
| 338258 0.02 mM | 187,835 | 383,737 | 180,382 | 37,946 |
| Day 3 | | | | |
| Control (Medium) | 314,958 | 1,078,791 | 407,094 | 61,222 |
| Ellipticine 2 mM | 70,824 | 234,414 | 110,776 | 33,129 |
| Ellipticine 0.2 mM | 331,985 | 1,117,650 | 355,794 | 62,648 |
| Ellipticine 0.02 mM | 268,420 | 1,243,718 | 390,151 | 66,571 |
| 69187 2 mM | 289 | 17,247 | 820 | 1,048 |
| 69187 0.2 mM | 256,789 | 511,726 | 216,515 | 45,134 |
| 69187 0.02 mM | 371,712 | 805,110 | 411,171 | 63,864 |
| 338258 2 mM | 366 | 23,807 | 871 | 172 |
| 338258 0.2 mM | 384 | 111,306 | 1,254 | 515 |
| 338258 0.02 mM | 297,927 | 780,458 | 302,609 | 43,097 |
| Day 4 | | | | |
| Control (Medium) | 841,705 | 2,635,851 | 874,138 | 118,375 |
| Ellipticine 2 mM | 44,913 | 308,480 | 126,944 | 40,656 |
| Ellipticine 0.2 mM | 877,082 | 2,568,444 | 774,806 | 106,989 |
| Ellipticine 0.02 mM | 802,063 | 2,718,974 | 898,067 | 115,083 |
| 69187 2 mM | 463 | 9,111 | 601 | 628 |
| 69187 0.2 mM | 506,725 | 1,187,656 | 311,641 | 69,867 |
| 69187 0.02 mM | 824,251 | 2,582,505 | 869,367 | 112,366 |
| 338258 2 mM | 478 | 19,546 | 234 | 183 |
| 338258 0.2 mM | 530 | 123,316 | 1,026 | 291 |
| 338258 0.02 mM | 764,382 | 1,670,491 | 516,569 | 74,106 |
| Day 5 | | | | |
| Control (Medium) | 1,138,022 | 4,428,830 | 1,006,792 | 157,199 |
| Ellipticine 2 mM | 10,996 | 281,302 | 85,699 | 38,403 |
| Ellipticine 0.2 mM | 1,183,902 | 4,443,292 | 921,077 | 132,439 |
| Ellipticine 0.02 mM | 1,049,792 | 4,443,641 | 966,857 | 152,839 |
| 69187 2 mM | 375 | 1,745 | 213 | 144 |
| 69187 0.2 mM | 826,957 | 1,595,148 | 379,475 | 90,555 |
| 69187 0.02 mM | 1,347,042 | 4,299,468 | 1,011,078 | 145,073 |
| 338258 2 mM | 299 | 5,483 | 104 | 104 |
| 338258 0.2 mM | 376 | 64,052 | 321 | 101 |
| 338258 0.02 mM | 982,255 | 3,108,577 | 777,427 | 86,287 |

Furthermore, the present invention also demonstrates that the ellipticine derivatives were cytotoxic without possible collateral effects on normal cells. In order to demonstrate this, the effect of EPED3 on the cell growth of fetal bone derived normal mesenchymal cell was examined. The cell viability was tested every day for a period of five days. It was observed that these cells were not killed by the concentrations that were otherwise toxic to the myeloma cells.

Additionally, the effect of EPED3 was also compared to other known anti-neoplastic agents such as Etoposide (VP-16-213) and Adriamycin® (Doxorubicin Hydrochloride). Etoposide is an antitumor agent that complexes with topoisomerase II and DNA to enhance double-stranded and single-stranded cleavage of DNA and reversibly inhibit religation. It blocks the cell-cycle in S-phase and G2 phase, induces apoptosis in normal and tumor cell lines, inhibits synthesis of the oncoprotein, Mdm2 and induces apoptosis in tumor lines that overexpress Mdm2. Adriamycin® is a cytotoxic, anthracycline antibiotic used in antimitotic chemotherapy. It is infused intravenously to treat neoplastic diseases such as acute lymphoblastic leukemia, Wilms' tumor, soft tissue and osteogenic sarcomas, Hodgkin's disease, non-Hodgkin's lymphomas, Ewing's sarcoma and bronchogenic, genitourinary, breast and thyroid carcinoma (IARC 1976). Both these drugs are also used to treat myeloma. It was observed that the toxic effect of EPED3 was immediate compared to the other known anti-neoplastic agents.

In addition to the effect on myeloma cells, the present invention also demonstrated that EPED3 was effective against non-myeloma cell lines such as HEL, HL-60, k562, MEG01, THP1, Hela, g401, du145, sw480, sw620, saoS2 and MG63. Thus, EPED3 was a potent cell growth inhibitor. In summary, the preliminary data discussed supra demonstrated that the Ellipticine derivatives were potent inhibitors of myeloma and non-myeloma cancer cell growth. However, these derivatives did not affect the growth of normal cells. In comparison to the known anti-neoplastic agents, EPED3 inhibited the growth of myeloma cells much earlier than the anti-neoplastic agents that were tested. Hence, these Ellipticine derivatives could be used to inhibit the growth of myeloma and no-myeloma cancer cells.

The most remarkable genetic abnormalities identified in myeloma have been chromosome 13 deletion (Shaughnessy et al., 2000; Desikan et al., 2000), t(4;14) translocation, and jumping segmental duplications of chromosome arm 1q21 (Sawyer et al., 2005). These aberrations are associated with lower event-free and overall survival and shorter complete remission duration, as well as a significantly lower therapeutic response in patients receiving high-dose therapy and single- or double autologous stem cell transplants. Although most myeloma cell lines were initially cultivated from peripheral blood (e.g., L363, LP-1, OPM2, RPMI 8226, U266) or pleural effusion (e.g., ARK, ARP1, CAG, EJM, H929), all encode the common genetic mutations that exist in primary myeloma.

All myeloma cell lines used herein were spontaneously insensitive to many common anti-myeloma agents, which corresponded with U266 cells response to high doses of dexamethasone and VP-16-213 (FIG. 12). In addition, in vitro selection can be used to induce acquired drug resistances commonly seen in patients (Hazlehurst et al., 1999). The mutations conferring drug resistance can be categorized by their ability to limit uptake and enhance efflux of therapeutic agents (Gottesman, 2002). In a search for new therapeutic agents for multiple myeloma, the COMPARE algorithm was used to correlate global gene expression profile (GEP) with potency of anticancer compounds (Scherf et al., 2000).

For approximately 15 years, NCI's DTP has used a panel of 60 human tumor cell lines (NCI-60) derived from various tissues to screen potential anticancer agents for their ability to inhibit growth of multiple cancer cell lines (Monks et al., 1991). Early in this process, it was recognized that compounds with similar mechanisms had similar patterns of sensitivity (i.e., some cells are more sensitive to topoisomerase poisons, others less so). This led to development of the COMPARE algorithm (Paull et al., 1989), which compiled a list of compounds with patterns of growth inhibition similar to that of a “seed” compound supplied by an investigator. This approach can suggest potential mechanisms of action for novel compounds. Prominently, the NCI-60 panel has now been molecularly characterized (Holbeck, 2004; Sausville and Holbeck., 2004), which enables the COMPARE analysis to be extended to gene expression signatures.

The bone marrow microenvironment augments myelomagenesis and can protect myeloma cells from destruction when patients undergo systemic and supportive therapies. In vitro, via direct cell-cell adhesion or soluble factors generated by intracellular interactions, myeloma cells are reportedly well protected from chemotherapeutic agents (Nefedova et al., 2001). For instance, low-dose Velcade (4 nM) is a very effective anti-myeloma agent in the absence of stromal cells; however, when treating myeloma cells in co-culture with human fetal bone mesenchymal cells, myeloma cells exhibit excessive tolerance to Velcade (up to 500 nM), demonstrating more than 100-fold reduction in sensitivity (data not shown). Thus, the myeloma cell-mesenchymal cell co-culture system was used in the systematic screening of new synthetic compounds capable of overcoming the factors present in the bone marrow microenvironment that confound therapeutic efficacy. The results presented herein indicated that EPED3 effectively targets and kills myeloma cells, even in the presence of human mesenchymal cells, which demonstrates its promise as a new anti-cancer agent.

EPED3 was formed by modifying position 9 of ellipticine with a substitute radical of dimethyl amino-ethoxy. The radical preoccupies the hydroxylation position (9) of ellipticine (Chadwick ey al., 1978), which is likely to prevent its further metabolism and tremendously improved hydrophilicity of the compound. EPED3 retained ellipticine's high fluorescence spectral property (Sureau et al., 1993), absorbing ultraviolet energy at wavelengths 350 nm-495 nm and emitting at 660 nm as weak R-Phycoerythrin-Cyanine 5 signal.

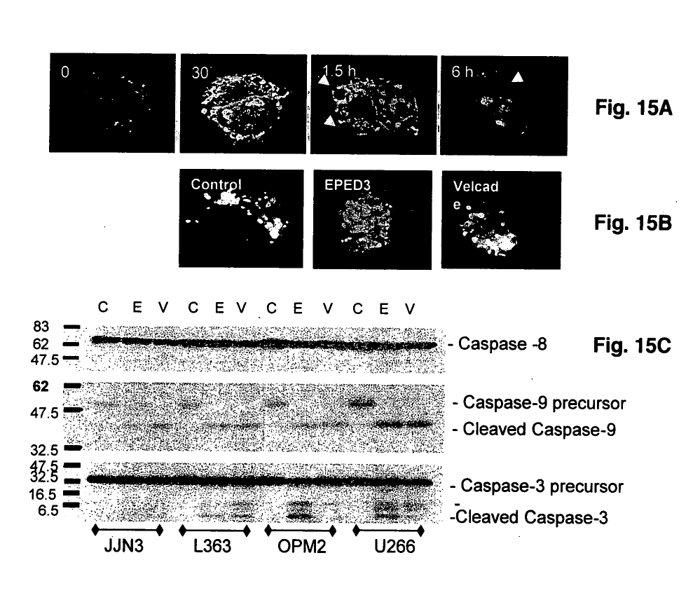

This spectral property proved advantageous for the fluorescence studies of EPED3 cytotoxicity. In vitro, uptake of EPED3 into cytoplasm is rapid and unrestrained; under the timed observations, EPED3 passively crossed the plasma membrane and evenly distributed throughout the cytosol within minutes (FIG. 15). This suggested that EPED3 was a lipophilic molecule entering cytoplasm independently of receptor- or ion channel-dependent mechanisms. Subsequent to its entry into cells, EPED3 instantly initiated enormous reduction of mitochondria and formation of large vacuoles (FIG. 15). It was likely that, like ellipticine, EPED3 was located in the mitochondrial inner membrane; this would provide an opportunity to disrupt the membrane potential by uncoupling mitochondrial oxidative phosphorylation, presumably through inhibition of the electron pathway of cytochrome c oxidase.

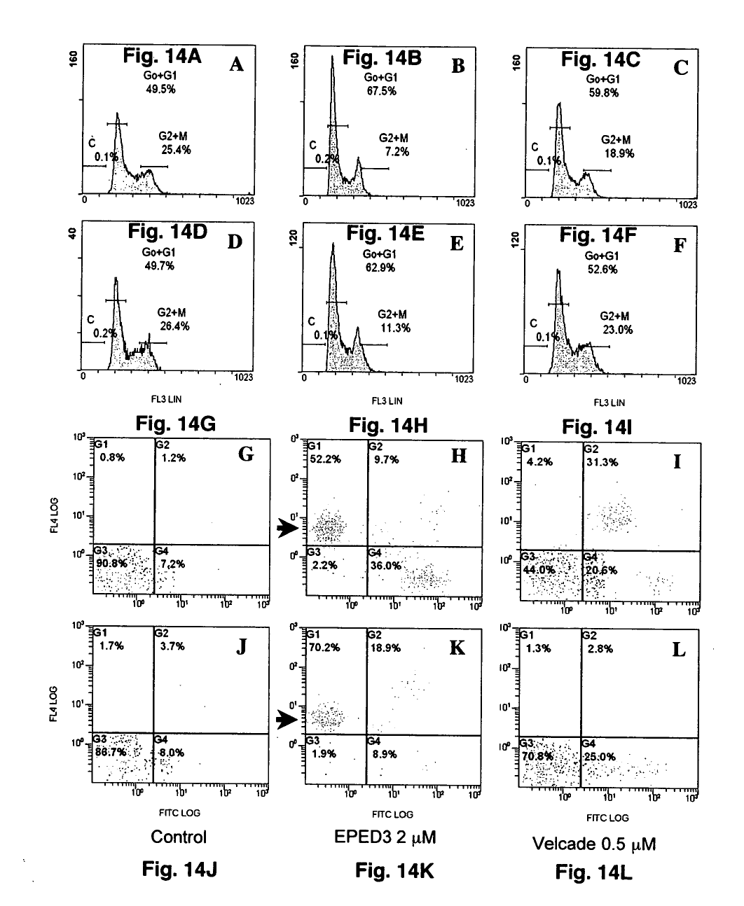

As rapidly as cytochrome c distributes into the cytoplasm upon EPED3 treatment, early apoptotic processes were detected using flow cytometry and Western blotting. The flow cytometry analyses demonstrated, through the level of Annexin-V staining, that 36% of myeloma cells were in early stages of apoptosis 12 hours after treatment with EPED3. Essentially, loss of the asymmetry of cell membrane phospholipids rendered phosphatidylserine on the surface and was measurable by Annexin-V affiliation. As the apoptosis process progressed, further loss of cell membrane integrity allowed DNA-specific dye, such as 7-Amino-actinomycin D (7-AAD), to enter the cell and incorporate into DNA strands; this allowed distinguishing between early and late apoptotic or necrotic cells (Ormerod et al., 1992; Schmid et al., 1994). Live cells remained 7-AAD_{negative}, apoptotic become 7-AAD_{dim}, and late apoptotic or necrotic cells appear 7-AAD_{bright} (FIG. 14). The increase of G₀+G₁-phase myeloma cells was another mark of cell growth arrest. Moreover, cleavage of caspase-9 and consequent activation of caspase-3 precursors, as demonstrated by Western blotting, marked initiation of the intrinsic pathway of programmed cell death. Diminished mitochondria resulting from EPED3 treatment also caused severe intracellular energy scarcity, which likely contributed to cell cycle arrest. The CellTiter-Glo Luminescent assay, which determined the amount of ATP in viable cells' metabolic activity, showed that EPED3 treatment resulted in the lowest luminescence readings of all agents tested (FIG. 12), suggesting a lack of ATP in EPED3 treated cells. Overall, multiple lines of evidence point to reduced cell viability and simultaneous cell cycle arrest in myeloma cells treated with EPED3 in vitro.

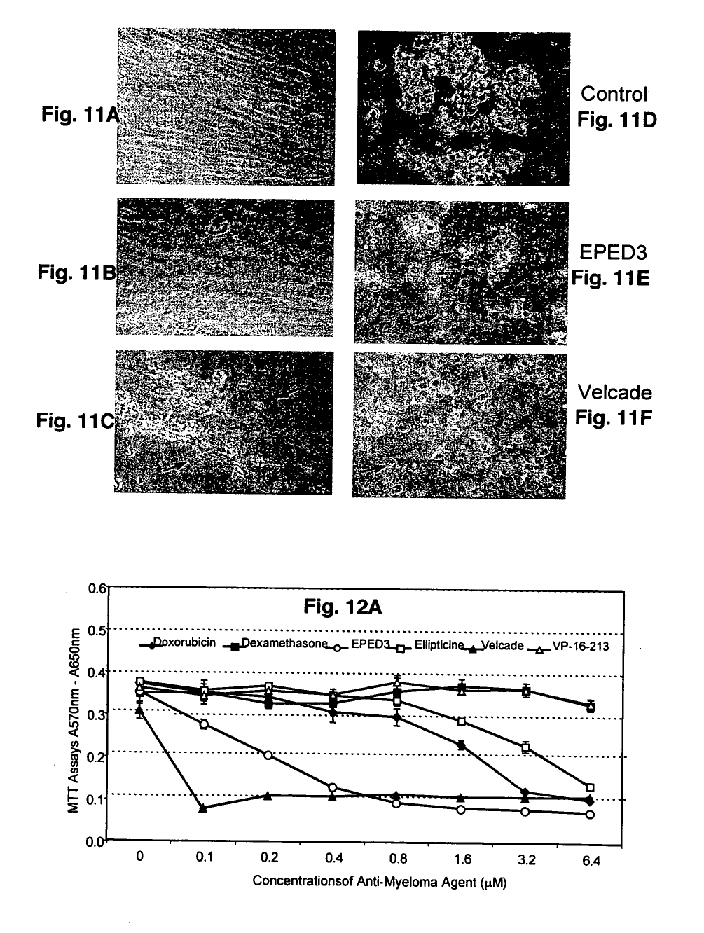

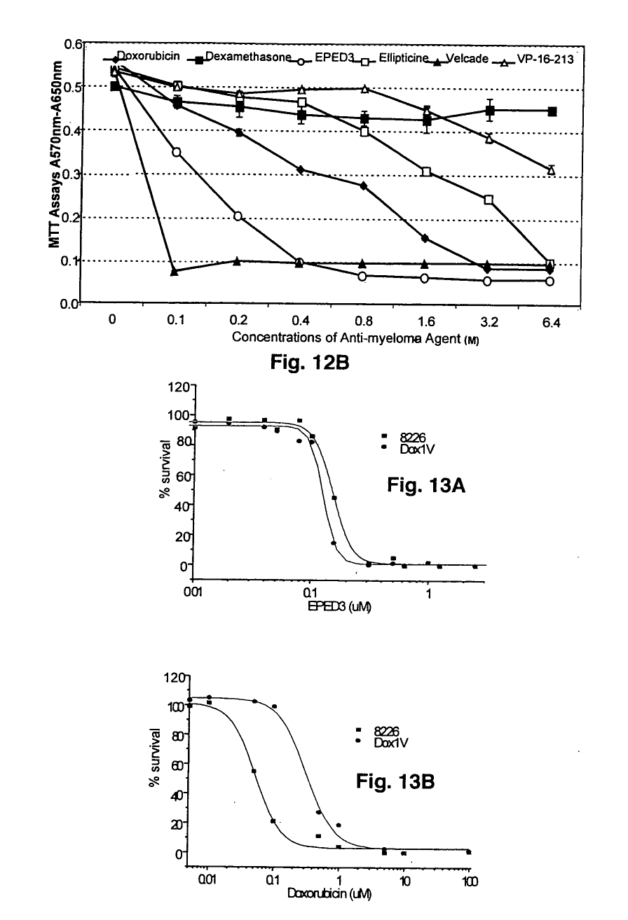

The data presented herein indicated that EPED3 could overcome acquired drug resistances that were selectively cultivated in myeloma cells to facilitate their tolerance of chemotherapeutic agents. The present invention used myeloma cell line 8226/Dox1V, a drug-resistant variant of RPMI 8226 cells that is characterized by reduced expression and activity of topo II, which confers insensitivity to a variety of topo II-dependent cytotoxicity. While initially categorized as a topo II inhibitor, ellipticine has now been shown to localize in cytoplasm and accumulate in mitochondria (Chadwick et al., 1978). The studies of EPED3 cytotoxicity indicated that it, too, was unlikely to directly affect topo II function in myeloma cells. Instead, mitochondrial disruption appeared to be the result of instant impact by intracellular EPED3, and that was associated with immediate release of cytochrome c. Cell growth inhibition by DNA intercalation and stimulation of topo II-mediated DNA breakage was a protracted response even in cultured cells. VP-16-213 has shown such topo II inhibitor activity, and myeloma cell lines (n=12) had a much more delayed response to VP-16-213 than to EPED3, regardless of the concentration (FIG. 12). Furthermore, the 8226/Dox1V cells exhibited a high tolerance to Dox (FIG. 13), which functions through topo II-inhibition, but EPED3 treatment resulted in prompt, dose-dependent killing of myeloma cells. Celiptium (NSC 264137) and Detalliptinium (NSC 311152) are two ellipticine derivatives clinically administrated against breast cancer. Both ellipticines were tested, in vivo, for DNA cleavage activity in comparison to m-AMSA, Amsacrine—a putative topo II inhibitor. Although IC₅₀ dosages of those agents are in a close range, Celiptium and Detalliptinium were, however, showing 50 times less potent in DNA strand breakage than m-AMSA (Multon et al., 1989). This was in agreement with finding presented herein of lack of topo II-dependent function among ellipticine-derivatives. Other than topo II-resistance, EPED3 could overcome the acquired drug tolerance to Velcade in myeloma cells. The in vitro chronic exposure to Velcade, an inhibitor to proteasome function by targeting the chymotryptic-like site of the molecule (Lee and Gottesman, 1998), has elevated the tolerance level (>5 nM) to the drug in several myeloma cell lines. It was observed that such Velcade-tolerance did not apply to EPED3, while the cell death occurred at the same low dose of EPED3 as the treatments to those parental myeloma cells (data not shown).

In addition to screening NCI-60 cancer cell lines, the DTP also conducts in vivo toxicity screenings. EPED3 was given to P388 Leukemia-bearing CD2F1 (CDF1) mice (maximal dose, 200 mg/kg body weight) by intraperitoneal injection. At 50 mg/kg, five out of six animals survived EPED3 toxicity of for at least 5 days. The dosage was equal to approximately 4.5 mM. This preliminary study supports the promise of EPED3 as a new generation of anti-myeloma agents for future preclinical studies to develop tailored therapies for patients with myeloma.

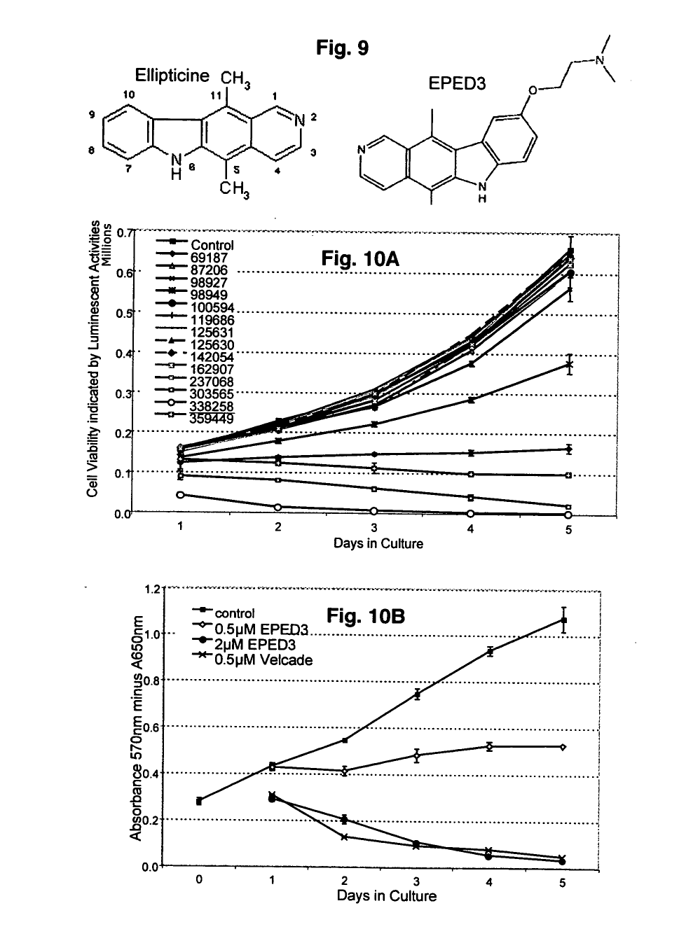

In summary, the present invention used NCI's COMPARE algorithm to identify ellipticines and several other synthetic compounds that showed a strong correlation between their GI50 for 60 human tumor cell lines (NCI-60) and the cells' expression levels of CKSiB. After investigating over 20 compounds, EPED3 was observed to be highly effective in killing myeloma cells. EPED3 is a highly hydrophilic derivative of ellipticine (FIG. 9), which is a hydrophobiccell-permeable alkaloid discovered in Apocyanaceae plants (Dalton et al., 1967). It was observed that EPED3 at nanomolar concentrations exhibited an extraordinary ability to kill all tested myeloma cell lines, including those sensitive to dexamethasone, etoposide (VP-16-213) and doxorubicin.

Furthermore, the present invention also investigated the mechanism of EPED3's cytotoxic effects on myeloma cells. Although ellipticine was reported binding to nucleic acids and acts as an inhibitor of topoisomerase-II (topo II) to stimulate topo II-mediated DNA breakage, the results presented herein indicated that EPED3 directly impacted cytoplasmic organelles, particularly targeting mitochondria, which subsequently triggered formation of apoptosomes and sequential activation of the cell death cascade. This mechanism was consistent with the ability of ellipticine to uncouple mitochondrial oxidative phosphorylation, presumably through its accumulation within the inner mitochondrial membrane and subsequent inhibition of the electron pathway of cytochrome c oxidase (Sureau et al., 1993). Other groups, however, have suggested different potential mechanisms for EPED3. For instance, it was implied that EPED3 was an inhibitor of RNA synthesis or an inducer of endoplasmic reticulum stress.

Additionally, since the present invention was directed to developing agents for treating multiple myeloma, the in vitro experiments were conducted in clinically relevant settings. The disease relies on dynamic interactions—both direct and indirect—between myeloma cells and bone marrow-derived stromal cells, and this synergy can efficiently protect malignant cells from drug-induced apoptosis (Nefedova et al., 2003). Hence, the in vitro EPED3 cytotoxicity studies were conducted on co-cultured myeloma and stromal cells to mimic the clinical setting and provide a protective environment to myeloma cells. The mesenchymal cells derived from human fetal bone were cultured with myeloma cells, both with and without direct contact between cell types. In these co-culture conditions, nanomolar concentrations of EPED3 resulted in rapid cell cycle arrest and massive apoptosis in myeloma cells. Furthermore, the present invention has also demonstrated the toxic effect of EPED3 on cancer cells other than myeloma cells. Thus, EPED3 is emerging as a novel agent in future tailored cancer treatments for individual patients' drug resistances.

In one embodiment of the rpesent invention, there is a metthod of treating myeloma in an individual, comprising administering a pharmacologically effective dose of ellipticine, a derivative thereof or a combination thereof to the individual. The ellipticine or the derivative thus administered may induce cell cycle arrest of myeloma cell, may induce apoptosis of myeloma cell, may overcome acquired drug resistance or a combination thereof without affecting the viability of normal cells. Examples of such derivatives of Ellipticine may include but are not limited to EPED3 (9-dimethyl amino-ethoxy elipticine), or NSC69187 (9-methoxy ellipticine). Furthermore, the type of individual that may benefit from such a method may be the one diagnosed with myeloma or the one resistant to drugs such as doxorubicin. In general, the route of administration of ellipticine, its derivative or a combination thereof may include but is not limited to oral, topical, intraocular, intranasal, parenteral, intravenousm intramuscular or subcutaneous route. Additionally, the dose range of the administered ellipticine, the derivative of ellipticine or its combination may be from about 0.01 mg/kg to about 500 mg/kg body weight of the individual.

In another embodiment of the present invention, there is a method of treating myeloma in an individual, comprising administering a pharmacologically effective dose of a topoisomerase II inhibitor, wherein said inhibitor induces cell cycle arrest of myeloma cell, induces apoptosis of myeloma cell, overcomes acquired drug resistance or a combination thereof without affecting the viability of normal cells, thereby treating myeloma in the individual. The topoisomerase inhibitor used in such a method may be ellipticine or its derivative. Furthermore, the examples of the ellipticine derivatives, the dose administered, route of administration and the type of individual benefitting from such a method is the same as discussed supra.

In yet another embodiment of the present invention, there is a method of inhibiting growth of a myeloma cell, comprising: contacting the myeloma cell with ellipticine, a derivative thereof or a combination thereof. Such a contact may inhibit myeloma cell growth by inducing cell cycle arrest, apoptosis or a combination thereof. Specifically, the apoptosis may be induced by activation of caspase 9. Additionally, the examples of derivatives that may be used in such a method is the same as discussed supra. Furthermore, the examples of myeloma cell may include but are not limited to ARP1, CAG, L363, MM144, OCI-my5, OPM2 or U266.

In another embodiment of the present invention, there is a method of inducing apoptosis of a myeloma cell, comprising contacting the myeloma cell with ellipticine, a derivative thereof or a combination thereof such that the contact activates caspase-9, thereby inducing apoptosis of the myeloma cell. Examples of the derivatives and the cells that may be used in such a method is the same as discussed supra.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. As used herein, the term “contacting” refers to any suitable method of bringing the sample into contact with the ellipticine, its derivative or a combination thereof. In vitro or ex vivo may be achieved by exposing the above-mentioned cell to the composition in a suitable medium.

The compounds described herein can be administered independently, either systemically or locally, by any method standard in the art. Dosage formulations of the composition described herein may comprise conventional non-toxic, physiologically or pharmaceutically acceptable carriers or vehicles suitable for the method of administration and are well known to an individual having ordinary skill in this art.

The composition described herein may be administered independently or in combination with any other anti-neoplastic or chemotherapeutic agent and may comprise one or more administrations to achieve, maintain or improve upon a therapeutic effect. It is well within the skill of an artisan to determine dosage or whether a suitable dosage of the composition comprises a single administered dose or multiple administered doses. An appropriate dosage depends on the subject's health, the inhibition of myeloma cell growth either by inducing cell cycle arrest or by inducing apoptosis, the route of administration and the formulation used.

The following examples are given for the purpose of illustrating various embodiments of the invention and are not meant to limit the present invention in any fashion:

### EXAMPLE 1

Preparation of Cell Cultures

All experiments were carried out in 96-well tissue culture plates. Cell counts were made for all cell lines using a hemacytometer. The cells were diluted to 20,000 cells/ml of medium. An aliquot of 50 μl was seeded for final cell counts at 1,000 cells/well.

### EXAMPLE 2

Sample Preparation

All compounds to be tested were originally dissolved in 100% DMSO at the concentration of 1 mg/ml. In the initial pilot dosage tests, the final concentrations of each compound were set at 5, 2.5. 1 and 0.5 μg/ml.

### EXAMPLE 3

Cell Viability Assay

Cell Titer-Glo luminescent cell viability assay kit from Promega Co (Madison, Wis.) was used to determine cell growth inhibition by the various compounds tested. The kit determines cell viability by quantifying the amount of ATP present in the cell culture medium. Presence of ATP signals the presence of metabolically active cells (3). The intensity of luminescence was measured by a computerized luminometer (Promega, Co. Madison, Wis.). Each well was read five times within 30 minutes of adding Cell Titer-Glo reagent mixture.

### EXAMPLE 4

Cell Growth Inhibition Assay and Dosage Efficacy Test

Cells were contacted with the antineoplastic agents in ninety six well culture plates. The concentration of each neoplastic agent was maintained a constant. A cell culture medium control and 0.5% DMSO control were maintained in each set of experiments. The cell viability was tested every day using the CellTiter-Glo luminescent cell viability kit from Promega Co. (Madison, Wis.) for a period of five days.

Eight myeloma cell lines were contacted with the antineoplastic agents to be tested in ninety six well culture plates. The final concentrations of each antineoplastic agent were set at 5, 2.5, 1 and 0.5 μg/ml. A cell culture medium control and 0.5% DMSO control were maintained in each set of experiments. The cell viability was tested every day using the CellTiter-Glo luminescent cell viability kit from Promega Co. (Madison, Wis.) for a period of five days.

### EXAMPLE 5

Effect on Cell Viability

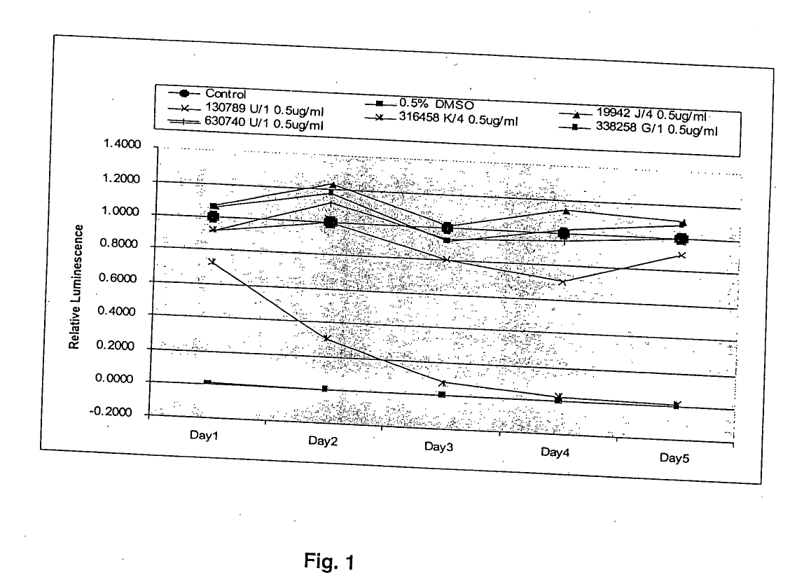

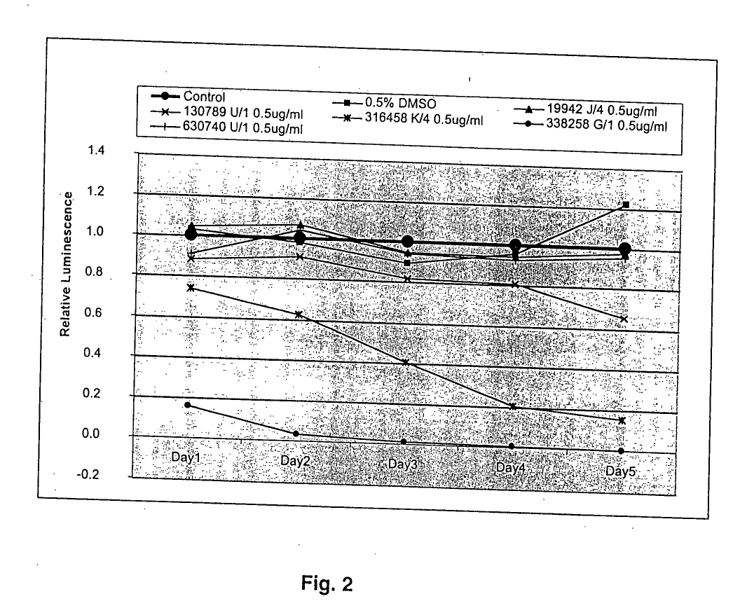

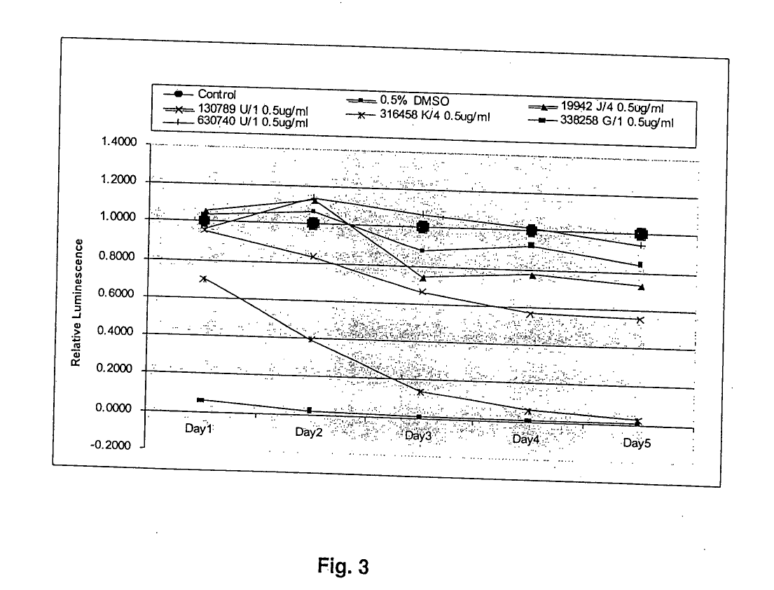

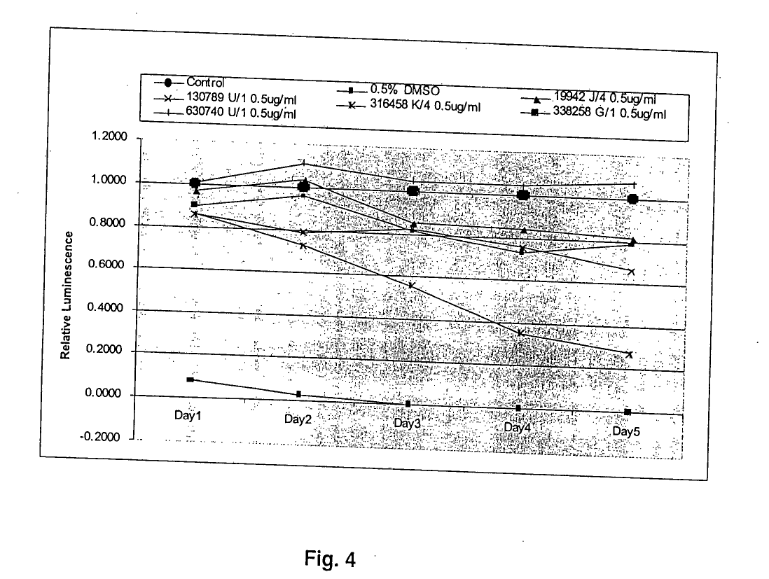

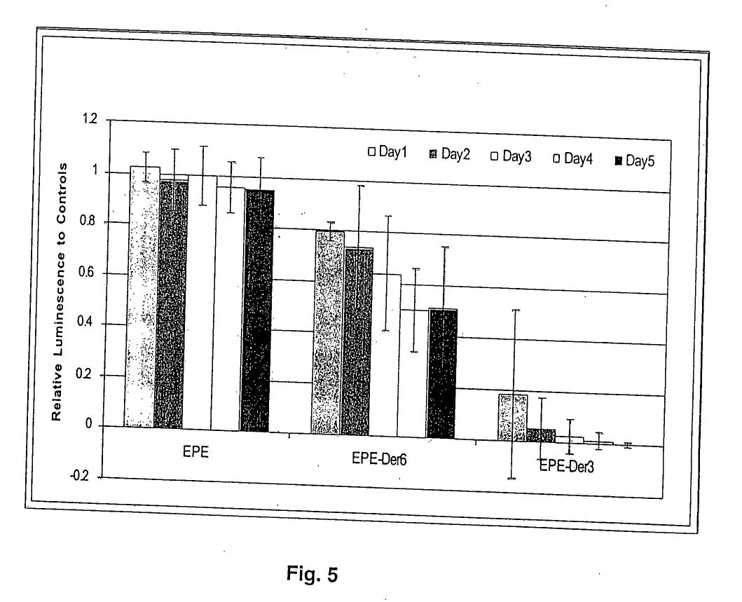

The CellTiter-Glo Luminscent Assay showed no significant difference in cell viability in the control (medium alone) and medium+DMSO (5%). Additionally, of the compounds that were tested, NSC 338258 showed a significant anti-myeloma effect on all 4 myeloma cell lines (FIGS. 1, 2, 3, 4). Furthermore, this effect was more than other compounds that were tested

### EXAMPLE 6

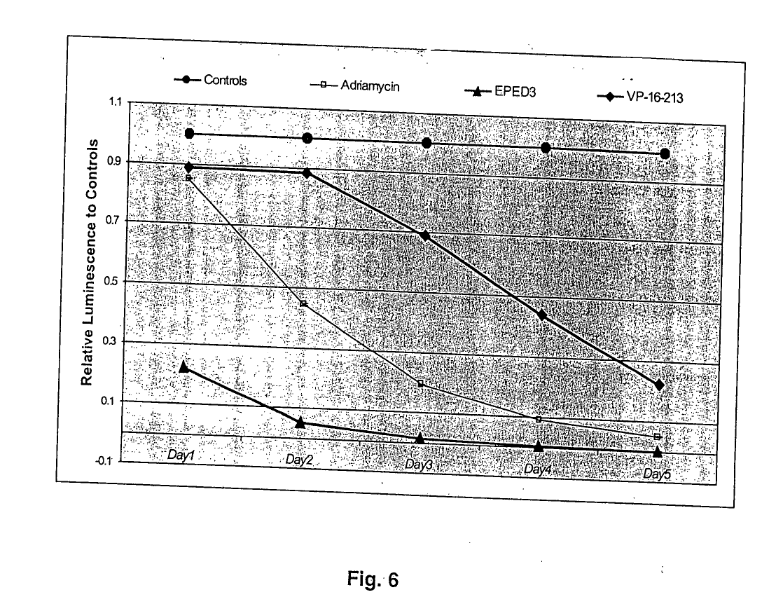

Comparative study of NSC 338258 with existing anti-myeloma drugs Myeloma cell lines were exposed to NSC 338258 and existing anti myeloma drugs, Adriamycin® (Doxorubicin Hydrochloride) and Etoposide (VP-16-213) in ninety six well culture plates. The concentration of each compound was set at 0.2 mM. The cell viability was tested every day using the CellTiter-Glo luminescent cell viability kit from Promega Co. (Madison, Wis.) for a period of five days. In vitro results showed that NSC 338258 had immediate inhibition of cell proliferation within the first 24 hours. The cell growth was near zero in the following days (day 4 onwards; FIG. 6). It was also observed that 12 myeloma cell lines (Ark, BW288, CAG, Delta47, DF15, JJN3, kms11, L363, opm2, RPMI8226, U266 and XG1) responded to the same dosage (0.2 μM) of NSC 338258, VP-16-213 and Adriamycin in 5 days.

### EXAMPLE 7

Selectivity of NSC 338258

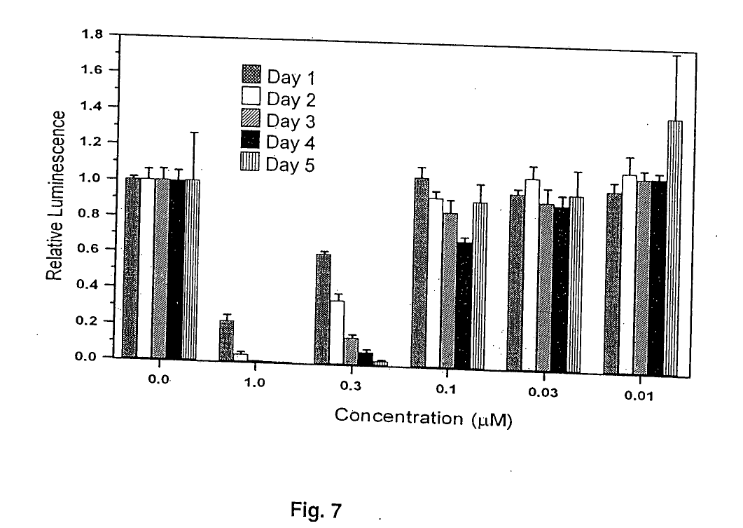

Fetal bone mesenchymal cells were contacted with NSC 338258 in ninety six well culture plates. The concentrations of NSC 338258 were set at 0.01, 0.03. 0.1, 0.3 and 1.0 mM (FIG. 7). The cell viability was tested every day using the CellTiter-Glo luminescent cell viability kit from Promega Co. (Madison, Wis.) for a period of five days.

### EXAMPLE 8

Overall Cell Growth Inhibition of NSC 338258

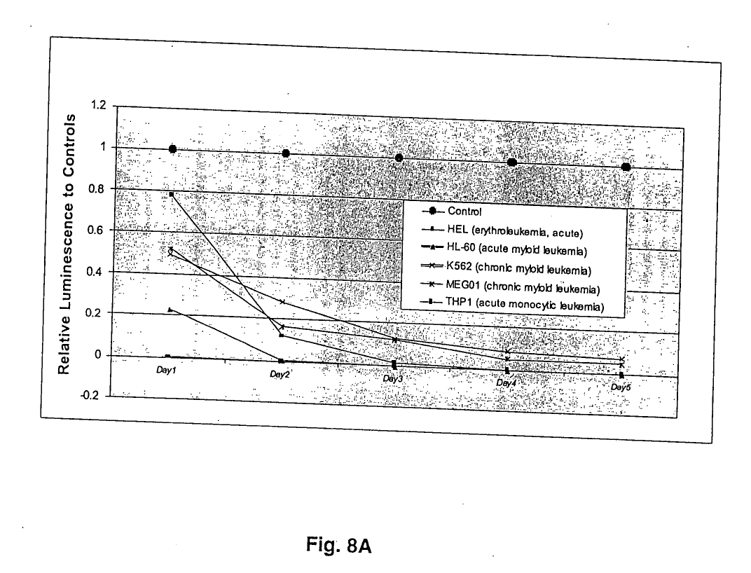

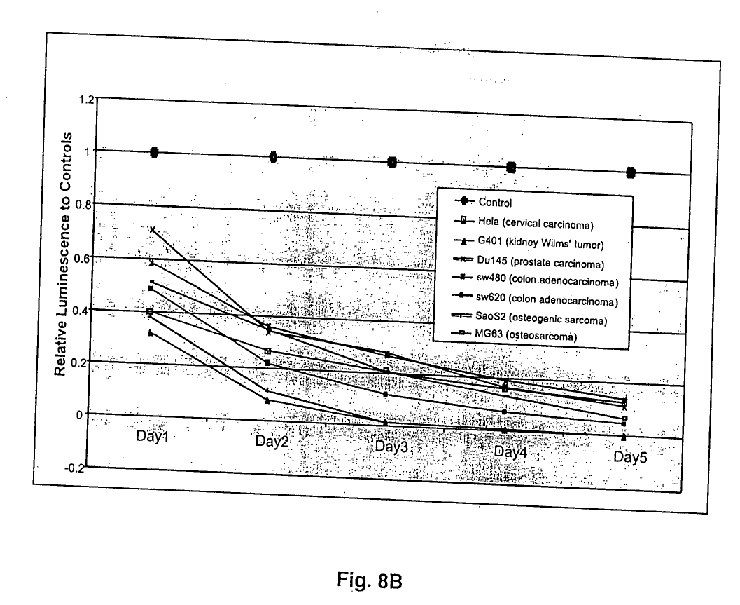

To test the overall cell growth inhibition of NSC 338258, twelve non myeloma cancer cell lines, HEL, HL-60, k562, MEG01, THP1, Hela, G401, Du145, sw480, sw620, saoS2, and MG63, were exposed to the compound in 96 well cell culture plates for a period of five days. A cell culture medium control and 0.5% DMSO control were maintained in each set of experiments. The cell viability was tested every day using the CellTiter-Glo luminescent cell viability kit from Promega Co. (Madison, Wis.) for a period of five days (FIGS. 8A-8B).

### EXAMPLE 9

Myeloma Cell Lines and Culture

Human myeloma cell lines (OPM-2, RPMI 8226, and U266) were purchased from the American Type Culture Collection, Manassas, Va.). Except ARK, ARP1, and CAG myeloma cell lines, most of the myeloma cell lines were provided by Michael Kuehl, Md. (Genetics Department, Medicine Branch, National Cancer Institute, Bethesda, Md.). The RPMI 8226/Dox1V drug-resistant variant was the gift of William S. Dalton (H. Lee Moffitt Cancer Center, Tampa, Fla.) and is maintained under chronic drug pressure (1×10⁻⁸ M doxorubicin [Dox] and 10 μg/ml verapamil [both from Sigma-Aldrich, St. Louis, Mo.] weekly).

All cell lines were maintained in RPMI-1640 medium supplemented with 10% FBS, 100 unit/ml of penicillin/streptomycin, 2 mM of L-glutamine, and 1 mM of sodium pyruvate (Invitrogen Co, Carlsbad, Calif.). Cells were incubated at 37° C. with 5% CO₂. Cell viability was determined using hemacytometer with trypan blue stain (Invitrogen, Co, Carlsbad, Calif.).

### EXAMPLE 10

DTP Compounds and Other Agents

All the DTP compounds were obtained from the Developmental Therapeutic Program of National Cancer Institute (NCI). Doxorubicin (Dox), Ellipticine, and Etoposide (VP-16-213) were purchased from Sigma (St. Louis, Mo.). Velcade (bortezomib or PS-341) was provided by Millennium Pharmaceuticals Inc (Cambridge, Mass.). Dexamethasone was purchased from Elkins-Sinn (Cherry Hill, N.J.).

### EXAMPLE 11

Co-Culture System

The monolayer human fetal bone mesenchymal cells were established from live bone chips (Advanced Bioscience Resources Inc, Alameda, Calif.) in complete RPMI-1640 medium. The medium was the same as described above. The cells were trypsinized and seeded in 6-well plates, and then allowed to reach 75% confluence. For indirect contact co-culture, myeloma cells were suspended in the TC culture inserts with 3.0-mm pore size track-etched polyethylene terephthalate (PET) membranes (Becton Dickson Labware, Franklin Lakes, N.J.) at 10⁶ cells/ml of total volume of medium (10 ml/well), while the human fetal bone mesenchymal cells were adhering to the bottom of the plates. Anti-myeloma agents were added individually at desired concentrations.

For direct-contact co-culture, human mesenchymal cells were seeded in 96-well plates with 50 μl of medium, and then allowed to reach 50% confluence. Myeloma cells (25-μl aliquot) were added to wells with mesenchymal cells to reach a final concentration of 5,000 myeloma cells/well. To each plate, 75 μl of medium contains non-drug, NSC 338258 (EPED3), or Velcade at desired concentrations.

### EXAMPLE 12

In Vitro Screening of Compounds Obtained from NCI's Developmental Therapeutics Program

Myeloma cell lines were maintained at >90% viability at 37° C. and 5% CO₂. Cells were diluted to 20,000 cells/ml in fresh medium. A 50-μl aliquot of cell suspension was seeded in 96-well tissue culture plates to final cell counts of 1,000 cells/well. All compounds obtained from NCI's Developmental Therapeutics Program (DTP compounds) were dissolved in 100% dimethylsulfoxide (DMSO) (Sigma-Aldrich, St. Louis, Mo.) at 1 mg/ml and further diluted in fresh medium to 1 μg/ml. Each compound (50-μl aliquots) was distributed to a well containing 1,000 myeloma cells. Each plate included controls with addition of only medium or only DMSO (0.5% final concentration). All experiments were conducted in triplicate.

Using CellTiter-Glo Luminescent Assay kit (Promega, Madison, Wis.) according to manufacturer's instructions, cell viability was assayed every 24 hours after addition of DTP compound for 5 days. Briefly, the CellTiter-Glo substrate was dissolved in buffer, and 100 μl was added to each well. The intensity of luminescence was measured by a computerized luminometer (Promega, Madison, Wis.); each well was read five times within 30 minutes. The statistic sampling distribution for each compound per cell line was based on sample of n=15 measurements.

### EXAMPLE 13

MTT Assay of Cell Proliferation

Myeloma cells were suspended in fresh medium at 2×11 cells/ml and seeded into 96-well plates in 50-μl aliquots (10,000 cells/well). After treating cells in each well with the appropriate compound (50-μl aliquots of 2× stock solution) and medium control, the methylthiazolyldiphenyl-tetrazolium bromide (MTT) assay kit (Promega, Madison, Wis.) was used to determine cell proliferation, according to manufacturer's instructions. Briefly, dye solution was added to each well (15 μl/well) and incubated for 4 hours at 37° C. and 5% CO₂; 100 μl of the solubilization solution/stop mix was then added. The plates were read at 570 nm in a plate reader, and the A570 nm values were corrected by A650 nm. All experiments were conducted in triplicate.

### EXAMPLE 14

Drug Resistance and Cytotoxicity Assay

The 8226/Dox1V cell line was established by chronic exposure to Dox in presence of P-glycoprotein inhibitor, verapamil and was characterized by reduced expression and activity of topo II (Futscher et al., 1993). The cell line demonstrated cross-resistance to other topo II poisons such as mitoxantrone and etoposide, but was not resistant to chemotherapeutic agents not known to target topo II, including melphalan, vincristine, cytarabine, or dexamethasone.

The 8226/Dox1V and its parental line, RPMI 8226, cells were plated in 96-well plates at 10,000 cells/well and incubated with serial dilutions of EPED3 or Dox, as previously described (Landowski et al., 1999). After 96 hours incubation at 37° C., MTT was added to each well at a final concentration of 20 μg/ml and incubated an additional 4 hours. Plates were centrifuged and the media replaced with 0.1 ml DMSO to solublize the formazan complex. Optical density was measured at 540 nm using a Bio-Tek microplate spectrophotometer. IC₅₀ values were calculated by linear regression analysis (Origins® Software Technologies, Inc.) of the log-linear plot for percent survival versus log drug concentration.

### EXAMPLE 15

Gene Expression Profiling Assays

The GeneChip Human Genome U133Plus 2.0 Array system (Affymetrix, Inc., Santa Clara, Calif.) was utilized to conduct comprehensive analysis of genome-wide expression profiles in primary myeloma cells. The erythrocyte-depleted fraction of bone marrow aspirates were sorted for CD138+myeloma plasma cells using the AutoMACS magnetic cell separating technique (Miltenyi Biotec, Auburn, Calif.). The previously described cDNA microarray standard protocol was used (Zhan et al., 2003).

### EXAMPLE 16

Flow Cytometry Analyses

Cell cycle arrest was measured using DNA-Prep Coulter Reagents kit (Beckman Coulter, Inc., Miami, Fla.). Myeloma cells were harvested from co-culture inserts and washed in phosphate buffered saline (PBS); 5×10⁵ cells were stained according to the manufacturer's instructions. Samples were analyzed using EPICS XL-MCL flowcytometer equipped with EXPO32 ADC software (Beckman Coulter, Inc.).

Apoptosis was detected using the Annexin V-fluorescein isothiocyanate (FITC)/7-AAD kit (Beckman Coulter, Inc.). Myeloma cells were harvested from co-culture inserts and washed in PBS; 5×10⁵ cells were incubated on ice with 100 μl of binding buffer (10 μl Annexin V-FITC and 20 μl of 7-AAD) for 15 minutes. Samples were analyzed using EPICS XL-MCL flowcytometer equipped with EXPO32 ADC software (Beckman Coulter, Inc.).

### EXAMPLE 17

Mitochondria/Cytochrome c Staining

Myeloma cells were co-cultured with human fetal bone mesenchymal cells in the TC inserts. The cells were treated with individual antimyeloma agents at desired concentrations. An aliquot containing 10⁶ cells was transferred to 24-well plate and mixed with MitoTracker Red CMXRos (Invitrogen) at 50 nM final concentration. After a 15-minute incubation, cells were collected and washed with PBS and resuspended at 5×10⁵ cells/ml for slide adhesion by the Cytospinning method. The slides were fixed in 3.7% formaldehyde/PBS for 15 minutes and rinsed in PBS; cells were permeabilized in ice-cold acetone for 5 minutes and rinsed in PBD (PBS with 0.1% NP-40) solution twice. Monoclonal mouse anti-human cytochrome c antibody (R&D Systems, Minneapolis, Minn.) was diluted (10 mM Na⁺ PO₄ [pH 7.8], 0.15 M NaCl) 1:500 and added to each spot. After 15 minutes, slides were washed twice in PBD. The FITC-conjugated goat anti-mouse IgG H+L chain (GAM) (BD Biosciences, San Diago, Calif.) was added, and slides were incubated for 15 minutes. Finally, to stain DNA as a marker of cell nuclei, slides were stained with 0.1 μg/ml of 4′,6-diamidino-2-phenylindole (Sigma-Aldrich) in PBS and mounted with antifade solution (Invitrogen). Images were captured using the Genetic Station with red (for mitochondrial staining), green (for Cytochrome C staining), and blue (for nuclear staining) single bandpass filter set (Abbot Vysis, Co., Downers Grove, Ill.).

### EXAMPLE 18

Western Blotting and Detection

Myeloma cell lines were indirectly co-cultured with human mesenchymal cells as described above. EPED3 and Velcade were added to co-culture to final concentrations of 2 μM and 0.5 μM, respectively. After 6 hours of treatment, myeloma cells were harvested from the inserts, washed twice in PBS, and resuspended in protein extraction buffer (PBS, 10 μg/ml aprotinin, 10 μg/ml leupeptin, and 1 mM phenylmethylsulfonyl fluoride) to a final concentration of 10⁴ cells/μL. Cell lysates were prepared by three cycles of freezing in liquid nitrogen followed by thawing at 37° C. Protein concentration was determined by spectrophotometry (NanoDrop Technologies, Wilmington, Del.).

For sodium dodecyl-polyacrylamide gel elctrophoresis (SDS-PAGE), 100 μg of protein extract was mixed with NuPAGE LDS sample buffer (Invitrogen) and loaded into each lane of the gel (10%-15% polyacrylamide separating gel, 4% stacking gel). After electrophoresis, protein was transferred to Hybond ECL nitrocellulose membrane (Amersham Pharmacia Biotech, Piscataway, N.J.). The primary antibodies (goat anti-human caspase-3, -8, and -9 polyclonal antibodies) were (R&D Systems) detected with WesternBreeze kit (Invitrogen).

### EXAMPLE 19

EPED3 Demonstrated Significant Killing Activities Among all Tested DTP Compounds, and this Antimyeloma Efficacy cannot be Prevented by Stromal Cells in the Co-Culture System

Amplification and overexpression of CKS1B has been linked with de novo high-risk myeloma (Shaughnessy, 2005), and this putative oncogene maps to chromosome arm 1q21, which has high frequency of genomic instability in multiple myeloma (Sawyer et al., 2005). The COMPARE algorithm was used to query growth inhibition data in DTP's open database of approximately 44,000 synthetic compounds to identify potential anticancer compounds with higher potency in cells with increased expression of CKS1B. GI₅₀ values—the concentration that causes 50% growth inhibition—were used as selection criteria for this analysis. A substantial number of selected compounds had significant Pearson Correlation Coefficients (PCCs) (table 2). Among the compounds with a high PCC, those with a high standard deviation (SD), which measures the degree of difference in responses of cell lines to the compound of interest were examined. This group of compounds comprised of several derivatives of ellipticine.

TABLE 2
The PCC of Ellipticines ranked by killing acivities to NCI-60 cancer panel.

| | | | High | Mean | # cell | Std | # of | |
|---|---|---|---|---|---|---|---|---|
| Rank* | NSC** | Compound Name | Conc.^{#} | GI₅₀^{§} | lines^{∫} | Dev\hat{ } | Exp^{γ} | PCC^{✓} |
| 1 | 636859 | Cuspidatin C | −4 | −4.46 | 41 | 0.62 | 1 | 0.635 |
| 2 | 630740 | | −4 | −4.26 | 46 | 0.44 | 1 | 0.624 |
| 3 | 699959 | | −4 | −4.53 | 51 | 0.49 | 1 | 0.619 |
| 4 | 638280 | | −4 | −4.37 | 48 | 0.51 | 2 | 0.601 |
| 5 | 338258 | Ellipticine, 9− | −4 | −6.57 | 47 | 0.49 | 6 | 0.591 |
| | | Dimethyl-Amino-Ethoxy | | | | | | |
| 6 | 627891 | | −4 | −5.43 | 47 | 0.42 | 1 | 0.590 |
| 7 | 637132 | ellipticinium analog | −4 | −6.09 | 45 | 0.43 | 1 | 0.586 |
| 8 | 19942 | Tenulin | −4 | −4.68 | 58 | 0.49 | 1 | 0.58 |
| 9 | 637126 | ellipticinium analog | −4 | −6.40 | 58 | 0.43 | 2 | 0.578 |
| 10 | 640531 | | −4 | −4.99 | 46 | 0.46 | 1 | 0.569 |
| 11 | 622693 | | −4 | −4.53 | 41 | 0.76 | 1 | 0.569 |
| 12 | 316458 | Neplanocin A | −4 | −5.43 | 59 | 0.72 | 2 | 0.566 |
| 13 | 632620 | ellipticinium analog | −4 | −6.52 | 42 | 0.51 | 1 | 0.561 |
| 14 | 684041 | | −4 | −4.11 | 52 | 0.58 | 1 | 0.556 |
| 15 | 645583 | | −4 | −4.17 | 48 | 0.49 | 1 | 0.552 |
| 16 | 661238 | | −4 | −4.47 | 58 | 0.43 | 2 | 0.550 |
| 17 | 130789 | | −4 | −4.96 | 59 | 0.49 | 2 | 0.550 |
| 18 | 69187 | Ellipticine, 9-methoxy | −4 | −6.35 | 47 | 0.45 | 6 | 0.547 |

*Compounds are ranked from highest to lowest Pearson's correlation coefficient.
**NSC: NCI's identification for the compound.
^{#}Highest concentration tested, in log¹⁰ molar; tests run with 10-fold dilutions over a 5 log range (e.g., if high concentration is 10⁻⁴M, the lowest concentration will be 10⁻⁸M).
^{§}Average across the cell lines of the concentration at which growth is inhibited 50% relative to no compound.
^{∫}Nnumber of cell lines used to calculate correlation; calculation can only look at those cell lines that were included in both measurements (CKS1B and this compound)
\hat{ }Reports variability of response of individual cell line to a given compound (e.g., variability = 0 if a compound has equal activity in all cell lines).
^{γ}Number of times this compound was screened at the high concentration.
^{✓}PCC (Pearson's correlation coefficient): equals 1 for perfect match, −1 for perfect mirror image, and 0 for none.

Initial screening for effectiveness of DTP agents was set with four myeloma cell lines, ARK, KMS11, RPMI 8226, and U266. The DTP compounds: (NSC) 19942, 69187, 70194, 87206, 98927, 98949, 100594, 113237, 119686, 125630, 125631, 130789, 142054, 162907, 176327, 237068, 303565, 316458, 338258, 359449, 630740, 640526, 640531, and 665549 were added to the cells at final concentration of 0.5 mg/ml. A control of medium and 0.5% DMSO was also set in each panel. Cell viability was assayed every 24 hours over the following 5 days. Two compounds, NSC 338258 (EPED3) and NSC 69187, most significantly inhibited cell viability in four myeloma cell lines (discussed supra). Both these agents are derivatives of ellipticine. The ellipticine and their derivatives were examined further in myeloma-growth inhibition.

Other ellipticine derivatives from the DTP (at 0.5 mM final concentration) were used to screen the same four myeloma cell lines. The results indicated that EPED3 was the best among the tested ellipticine derivatives for its significant negative effects on myeloma cell growth (FIG. 10A). This suggested that the enhanced anti-neoplastic activity of ellipticine was due to the specific modifications made to produce EPED3.

The efficacy of EPED3 was then tested in seven myeloma cell lines (ARP1, CAG, L363, MM144, OCI-myS, OPM2, and U266) in co-culture with direct contact with newly established human fetal bone mesenchymal cells to assess their sensitivity to EPED3 in the presence of protective stromal cells. During the 5-day course of co-culture, exposure to EPED3 resulted in decreased proliferation of myeloma cells, even in the presence of mesenchymal cells (FIG. 10B); linear regression analysis indicated that the efficacy of 2 mM EPED3 with 0.5 mM Velcade (r²≧0.92, p=0.002).

Myeloma cells were also co-cultured with human fetal bone mesenchymal cells. JJN3, L363, OPM2, and U266 cells were exposed to EPED3 (2 mM) or Velcade (0.5 mM) in co-culture without direct contact with stromal cells. Cell viability was assayed every 24 hours with CellTiter-Glo reagent in following 6 days. Myeloma cell destruction was observed with both EPED3 and Velcade treatment; linear regression analysis indicted that the anti-myeloma efficacy of EPED3 highly significant associated with Velcade-treatment, (r²≧0.98, p<0.0001; Table 3) in all four myeloma cell lines tested. Importantly, however, severe destruction of the mesenchymal cells was observed under Velcade treatment, while such morphological damage was not seen under EPED3 treatment (FIG. 11).

TABLE 3
Linear Regression Analysis of viability of four
Myeloma Cell lines cocultured with FBMC after
EPED3 or Velcade treatment for 6 days

| EPED3 (2 μM) treatment vs. | |
|---|---|
| Control | Velcade (0.5 μM) |

| Cell lines | r² | p-value | r² | p-value |
|---|---|---|---|---|
| JJN3 | 0.27 | 0.191 | 0.99 | <0.0001 |
| L363 | 0.27 | 0.183 | 0.98 | <0.0001 |
| OPM2 | 0.23 | 0.225 | 0.99 | <0.0001 |
| U266 | 0.30 | 0.161 | 0.98 | <0.0001 |

### EXAMPLE 20

EPED3 is a Highly Effective Antimyeloma Agent that Inhibits Viability and Overcomes Acquired Drug Resistance

In vitro comparison of EPED3 to ellipticine and four anti-myeloma agents commonly used in clinical treatment regimens was carried out with four myeloma cell lines (JJN3, L363, OCI-MY5, and U266). EPED3, ellipticine, Dox, dexamethasome, VP-16-213, and Velcade were applied in a two-fold dilution series ranging from 0.1 to 6.4 μM. Cell proliferation was assessed using MTT assays 24 and 48 hours after initiating treatments. At 0.4 μM, EPED3 inhibited cell viability as effectively as Velcade; the results also indicated that EPED3 efficacy is dose dependent but not time dependent (FIG. 12).

In drug resistance studies, the efficacy of EPED3 with 8226/Dox1V cells and the parental line, RPMI 8226 cells was investigated. Both cell lines were treated with either EPED3 or Dox, and the MTT assays were used to assess cell growth. Linear regression analysis of 96-hour dose-response curves was used to compare cytotoxicity of EPED3 in 8226/Dox1V and RPMI 8226 cell lines. The mean IC₅₀ for RPMI 8226 was 150.3 nM (+40.6) and the mean IC₅₀ for 8226/Dox1V was 131.3 nM (±36.0) (p=0.7, FIG. 13A); thus, the cytotoxic activity of EPED3 for the drug-resistant myeloma cells was not significantly different from that for the parental cells. In distinct contrast, 8226/Dox1V demonstrated 7.3-fold resistance to Dox, a known topo II inhibitor, than did the parental RPMI 8226 cells (IC₅₀=293.5 nM vs. IC₅₀=40 nM, respectively; p<0.001) (FIG. 13B).

### EXAMPLE 21

EPED3 Induces Cell Cycle Arrest

To investigate the effects of EPED3 on apoptosis and cell cycle arrest of myeloma cells, U266 cells were exposed to EPED3 (2 μM) or Velcade (0.5 μM) in co-culture without direct contact with fetal human bone mesenchymal cells. Twelve hours after treatment, cells were harvested for using flow cytometry to detect ongoing apoptosis and cell cycle arrest.

The degree of cell death resulting from EPED3 treatment was marginal (36% apoptotic cells, 9.7% necrotic cells), compared to that from Velcade (20.6% apoptotic cells, 31.3% necrotic cells) (FIGS. 14H, 14I). However, cytotoxicity of both EPED3 and Velcade was blocked by addition of pan-caspase inhibitor, Z-VAD-FMK, which resulted in decreases of cell death with both EPED3 (8.9% apoptotic cells, 18.9% necrotic cells) and Velcade (25% apoptotic cells, 2.8% necrotic cells) treatments. This suggested that cytotoxicity of both agents was caspase dependent. However, the initiations of apoptotic pathway by EPED3 and Velcade were different.

The cell cycle arrest analysis was simultaneously performed to examine DNA helix in U266 cells at 12 hours. It was observed that 67.5% of the EPED3-treated population was in G₀+G₁ phase as opposed to 49.5% of the control population and 59.8% of Velcade-treated populations in G₀+G₁ phase. Furthermore, 7.2% of the EPED3-treated population was in G₂+M phase as opposed to 25.4% of the control and 18.9% of Velcade-treated populations in the G₂+M phase (FIG. 14A-14C). The linear regression of the data obtained 3, 6, 12, and 24 hours after initiation of treatment indicated that the increase in G₀+G₁ cell population and the decrease in G₂+M cell population under EPED3 did not correlate with the cell cycle changes resulting from Velcade treatment (r²=0.08 and r²=0.001 respectively; Table 4).

TABLE 4
Linear Regression Analysis of U266 cell-cycle
arrest when co-cultured with FBMC after EPED3
or Velcade treatment at 3, 6, 12, and 24 hour.

| | Effect of EPED3 (2 μM) vs. | | | |
|---|---|---|---|---|
| | Control | | Velcade (0.5_M) | |
| The Helix of DNA | r² | p-value | r² | p-value |
| Increases in G₀ + G₁ Peak | 0.78 | 0.11 | 0.08 | 0.71 |
| Decrease in G₂ + M Peak | 0.56 | 0.25 | 0.001 | 0.96 |

### EXAMPLE 22

EPED3-Induced Cell Death

Autofluorescence of EPED3 was used to visually examine its effects on myeloma cells. The EPED3 molecule exhibited fluorescent emission when exposed to ultraviolet light at ≦500 nm wavelength. Cells that ingested EPED3 were, therefore, discernible by the characteristic golden light emitted under fluorescence microscopy. When cells were treated with EPED3, it instantly permeated the cells and distributed throughout the cytosol (FIG. 15A). Six hours after treatment, large vacuoles were formed within the cells, indicating ongoing apoptosis. Disintegration of the plasma membrane, another hallmark of apoptosis, was visualized as intensity of EPED3 dimmed in the cytosol.

To further investigate effects of EPED3 on mitochondrial integrity and cytochrome c, treated U266 cells were immunohistochemically analyzed. Disruption of mitochondria in U266 cells treated with EPED3 was recognized by staining with MitoTracker Red CMXRos, and subsequent release of cytochrome c from the mitochondrial membrane into the cytosol was also detected immunohistochemically (FIG. 15B). In contrast, mitochondrial disruption was not seen in cells similarly treated with Velcade (FIG. 15B).

The activation of caspases involved the mitochondrial apoptotic pathway was also examined using Western blotting. JJN3, L363, OPM2, and U266 cells were co-cultured with human fetal bone mesenchymal cells without contact and treated with EPED3 or Velcade for 6 hours. Proteins were then extracted from the harvested cells and analyzed for activation of caspase-8, -9, and -3 (FIG. 15C). There was no obvious activation of caspase-8 in any myeloma cell line tested, which excluded the involvement of the extrinsic apoptotic pathway. On other hand, there was a strong indication of activation of caspase-9 in all myeloma cell lines. Furthermore, it was observed that caspase-3 was cleaved in treated cells (FIG. 15C), further indicating an activated intrinsic apoptotic process in triggering the proteolytic cascade.

### EXAMPLE 23

In Vivo Study in Mice and Rats

In addition to the invitro evaluation of the effect of Ellipticine and its derivatives, the pharmacokinetics, tissue distribution and toxicology of the compouds is examined in mice and rats.

(a) Synthesis of metabolite standards: Five gms of 9-Hydroxy DMAEE (NSC 338258 or EPED3) is prepared using a patented 5-step process. This provides 1 gm of the 9-OH metabolite and enough additional 9-OH to make the glucuronide and the sulfate by enzymatic synthesis. Additionally, [¹⁴C] EPED3 is also synthesized. A reverse-phase HPLC method is used to separate the parent compound and the three metabolite standards to be synthesized. It utilizes an Agilent Model 1100 or 1050 HPLC equipped with a UV and Radiomatic detector for the visualization of unlabeled and radiolabeled compounds, respectively.

(b) Elimination of Radiocarbon following intravenous administration of [¹⁴C] EPED3 (6 mg/Kg, 1×10⁶ DPM/mouse) to mice: 6 female SCID bg mice are caged individually in glass Roth Metabolism cages One of the mice serves as control and 5 are treated with the compound. One week prior to dosing, the mice are acclimated to caging and feed. Throughout the study period, the mice are provide with feed and water ad libitum. Urine, feces and CO₂ are collected separately for radioassay and metabolite profiling by HPLC. Excreta collection intervals are 0 (control), 6, 24, 48, 72 and 96 hours. Excreta collection vessels are maintained at ˜4° C. (ice-water bath). Concentration and percent of administered radioactivity are determined by direct liquid scintillation counting of urine aliquots and CO₂ trapping solution (10% KOH) aliquots and by combustion of feces followed by LSC. HPLC analysis of metabolites are performed directly on urine samples. Urine from all 5 treated animals are pooled for each collection period.

(c) Pharmacokinetics and Tissue distribution of the compound (intravenous administration, 6 mg/Kg, 1×10⁶ DPM/mouse): 3 female SCID bg mice per time point are serially sacrificed for blood samples obtained by heart puncture and placed in heparinized tubes. The mice are acclimated to caging and feed for 1 week prior to dosing. The mice are provided with feed and water ad libitum throughout the study period. The blood samples are collected at 0, 5, 10, 20, 30, 45, 60, 120, 180, 240 minutes and 24 hours. The concentration is determined by combustion of 50 _L of whole blood followed by direct liquid scintillation counting (LSC). At time point 0 hrs, the mice are treated and serve as controls. HPLC analysis is performed on 0, 10, 30, 60, 120, 240-minute and 24-hour samples (all 3 animals blood pooled for analysis) to determine the metabolic profile. The following tissues are removed from the 60 and 240-minute animals for analysis of total ¹⁴C concentration (EPED3 equivalents) by combustion analysis and subsequent LSC: liver, kidneys, brain, heart, lungs, salivary gland, pancreas, adrenals, thyroid, thymus, lymph nodes, spleen, bone marrow, adipose, skeletal muscle, stomach, small intestine and large intestine. Contents are removed from intestines prior to analysis.

(d) Biliary excretion of [14C] EPED3 (i.v. administration 6 mg/Kg, 5×10⁶ DPM/rat), and metabolites by rats: 5 female Sprague-Dawley rats with canulated bile ducts are used for bile sample collection. Their carotid arteries are canulated for dose administration. The rats are acclimated to caging and feed for 1 week prior to dosing. The rats are provided with feed and water ad libitum throughout the study period. The samples are collected at 0, 5, 10, 20, 30, 45, 60, 120, 180, 240 minutes and 24 hours. At time 0, the rat will serve as the control. The concentration and the percent of dose is determined by direct liquid scintillation counting (LSC) of appropriate volume aliquots. The HPLC analysis is performed on 0, 10, 30, 60, 120, 240-minute and 24-hour samples (all 5 animals bile will be pooled for analysis) to determine the metabolic profile.

(e) Efficacy of EPED3 in kiling human myeloma in vivo:

The present invention also examines novel therapeutic approaches for myeloma using experimental animal models. The anti-myeloma efficacy of 9-(Dimethylaminoethoxy)-ellipticine is examined using a panel of myeloma cell lines engrafted in severe combined immunodeficient (SCID) mice. The 3 cell lines are infected with a lentivirus-containing luciferase. Luciferase-expressing myeloma cells can be traced in live animals using the Xenogen IVIS 200 luminescence imaging system. Six to 8 weeks old SCID mice are subcutaneously injected with 10⁷ myeloma cells. Tumor growth in live animals is monitored by measuring circulating level of monoclonal human immunoglobulins (hIg) in mice sera using ELISA (Yaccoby et al., 1998; Yang et al., 2002) and through imaging of tumor cells luminescence intensity. The tumor growth is detected within one week by ELISA and within 1-3 weeks by imaging.

Upon establishment of myeloma growth as determined by both methods, mice are randomly divided into 3 groups; one control and 2 treated with different doses of 9-(Dimethylaminoethoxy)-ellipticine (8 mice/group for each cell line). Mice are implanted with Alzet osmotic pumps delivering 0.25 μl of drug solution per hour for 28 days. The daily doses are 25 and 50 mg/kg/day. Control animals have pumps loaded with buffer (PBS) only. At the experiments' end, mice are subjected to imaging analysis, bled and sacrificed. Subcutaneous tumors are removed, weighed, histologically processed and internal organs are examined for potential damage. Through out the experimental period the mice are weighed and closely observed for potential side effects. It is contemplated that 9-(Dimethylaminoethoxy)-ellipticine effectively inhibits myeloma cell growth in vivo with no or minimal side effects.

The following references were cited herein:

Barlogie et al., Blood. 2004 Jan. 1; 103(1): 20-32.
Chadwick et al. Drug Metabolism & Disposition. 1978; 6; 5
Chesi et al. Nature Genetics. 1997; 16, 260-64.
Dalton et al., Aust J Chem 1967, 20; 2715-27.
Desikan et al., Blood 2000 Jun. 15; 95 (12): 4008-10.
Futscher et al. Analytical Biochemistry 1993 September 213 (2): 414-421.
Gottesman. Annual Review of Medicine. 2002, February (53): 615-627.
Hägg et al., Molecular Cancer Therapeutics. 2004, 3: 489-497
Hazlehurst et al., Cancer Research. 1999 Mar. 1; 59: 1021-28.
Holbeck S L. Eur J Cancer. 2004 April; 40 (6):785-93.
Huang et al., The Pharmacogenomics Journal. 2005, 5; 112-125.
Jagannath et al. British Journal of Hematology. 2004, 127: 165-72
Landowski et al., Blood. 1999 July; 94: 265-274.
Monks et al. J Natl Cancer Inst. 1991 Jun. 5; 83 (11):757-66.
Multon et al., Biochem Pharmacol. 1989 Jul. 1; 38(13): 2077-86.
Nefedova et al., Leukemia. June 2003, Vol 17 (6): 1175-82.
Ormerod et al., J Immunol Meth. 1992;153: 57-65.
Paull et al. J Natl Cancer Inst. 1989. Jul 19; 81 (14):1088-92.
Sausville and Holbeck. Eur J Cancer. 2004 November; 40 (17): 2544-9.
Sawyer et al., Genes Chromosomes Cancer. 2005 January; 42(1): 95-106.
Scherf et al. Nature Genetics. 2000; 24: 236-244.
Schmid et al., J Immunol Meth. 1994; 170: 145-57.
Shaughnessy et al.,. Blood. 2000 Aug. 15; 96 (4): 1505-11.
Shaughnessy et al. Blood. 2001 Jul. 1; 98(1): 217-23.
Shaughnessy Hematology. 2005; 10 Suppl 1:117-26.
Stiborova et al., Cancer Res. 2004 Nov. 15;64(22):8374-80.
Stiborova M et al. Biochem Pharmacol. 2001 Dec. 15;62(12):1675-84.
Sureau et al. Biophys J. 1993 November; 65(5): 1767-74
Sureau et al. Biophys J. 1993 November; 65(5): 1767-74.
Technical bulletin No. 288, Promega Co.
Zhan et al., Blood. 2003 Feb. 1;101(3):1128-40.

What is claimed is:

1. A method for treating myeloma in an individual, comprising:
administering a pharmacologically effective dose of ellipticine, a derivative thereof or a combination thereof to the individual,

2. The method of claim 1, wherein said ellipticine or the derivative thereof induces cell cycle arrest of myeloma cell, induces apoptosis of myeloma cell, overcomes acquired drug resistance or a combination thereof without affecting the viability of normal cells.

3. The method of claim 1, wherein said derivative of ellipticine is 9-dimethyl amino-ethoxy ellipticine or 9-methoxy ellipticine.

4. The method of claim 1, wherein said individual is diagnosed with myeloma or is resistant to drugs such as doxorubicin.

5. The method of claim 1, wherein said ellipticine, derivative thereof or combination thereof is administered via oral, topical, intraocular, intranasal, parenteral, intravenous, intramuscular or subcutaneous route.

6. The method of claim 1, wherein said ellipticine, derivative thereof or combination thereof is administered in a dose range of from about 0.01 mg/kg to about 500 mg/kg body weight of the individual.

7. A method of treating myeloma in an individual, comprising:
administering a topoisomerase II inhibitor, wherein said inhibitor induces cell cycle arrest of myeloma cell, induces apoptosis of myeloma cell, overcomes acquired drug resistance or a combination thereof without affecting the viability of normal cells, thereby treating myeloma in the individual.

8. The method of claim 7, wherein the inhibitor is ellipticine or a derivative thereof.

9. The method of claim 8, wherein the derivative of ellipticine is 9-dimethyl amino-ethoxy ellipticine or 9-methoxy ellipticine.

10. The method of claim 7, wherein said inhibitor is administered in a dose range of from about 0.01 mg/kg to about 500 mg/kg body weight of the individual.

11. The method of claim 7, wherein said inhibitor is administered via oral, topical, intraocular, intranasal, parenteral, intravenous, intramuscular or subcutaneous route.

12. The method of claim 7, wherein said individual is diagnosed with myeloma or is resistant to drugs such as doxorubicin.

13. A method inhibiting growth of a myeloma cell, comprising:
contacting the myeloma cell with ellipticine, a derivative thereof or a combination thereof.

14. The method of claim 13, wherein the ellipticine or the derivative thereof inhibits myeloma cell growth by inducing cell cycle arrest, apoptosis or a combination thereof.

15. The method of claim 14, wherein the apoptosis is induced by activation of caspase 9.

16. The method of claim 13, wherein said derivative is 9-dimethyl amino-ethoxy ellipticine or 9-methoxy ellipticine.

17. A method of inducing apoptosis of a myeloma cell, comprising:
contacting the myeloma cell with an ellipticine, a derivative thereof or a combination thereof, such that the contact activates caspase 9, thereby inducing apoptosis of the myeloma cell.

18. The method of claim 17, wherein said derivative is 9-dimethyl amino-ethoxy ellipticine or 9-methoxy ellipticine.
